(19) United States
(12) Patent Application Publication
(10) Pub. No.: US 2022/0214345 A1
(43) Pub. Date: Jul. 7, 2022

(54) COLORECTAL CANCER SCREENING EXAMINATION AND EARLY DETECTION METHOD

(71) Applicant: Deutsches Krebsforschungszentrum Sitftung des öffentlichen Rechts, Heidelberg (DE)

(72) Inventors: Petra SCHROTZ-KING, Heidelberg (DE); Megha BHARDWAJ, Sandhausen (DE); Hermann BRENNER, Wiesenbach (DE)

(73) Assignee: Deutsches Krebsforschungszentrum Sitftung des öffentlichen Rechts, Heidelberg (DE)

(21) Appl. No.: 17/608,457

(22) PCT Filed: May 8, 2020
(86) PCT No.: PCT/EP2020/062903

§ 371 Date: Nov. 2, 2021

(30) Foreign Application Priority Data
May 8, 2019 (EP) 19173318.7

(51) Int. Cl. G01N 33/574

(52) U.S. Cl. CPC G01N 33/57446; G01N 2333/65; G01N 2333/4742; G01N 2333/485; G01N 2333/918; G01N 2333/70582; G01N 2333/70596; G01N 2333/96433

(57) ABSTRACT

The present invention pertains to a new method for the diagnosis, prognosis, stratification and/or monitoring of a therapy, of cancer, preferably colorectal cancer (CRC), in a subject. The method is based on the determination of the level of a panel of least one, preferably 3, 4 and most preferably at least 5, protein biomarker selected from the group consisting of the protein biomarkers Amphiregulin (AREG), Carcinoembryonic antigen (CEA), Insulin like growth factor binding protein 2 (IGFBP2), Keratin, type I cytoskeletal 19 (KRT19), Mannan binding lectin serine protease 1 (MASP1), Osteopontin (OPN), Serum paraoxonase lactonase 3 (PON3) and Transferrin receptor protein 1 (TR), in the biological sample obtained from the subject. The new biomarker panel of the invention allows diagnosing and even stratifying various cancer diseases. Furthermore, provided are diagnostic kits for performing the non-invasive methods of the invention. Since the biomarker panel of the invention provides a statistically robust method independent of the protein detection technology used, and considering that the biomarker panel of the invention is detected in plasma samples of the subjects, the invention provides an early detection screening examination that may be applied to a larger population.

### FIELD OF THE INVENTION

The present invention pertains to a new method for the diagnosis, prognosis, stratification and/or monitoring of a therapy, of cancer, preferably colorectal cancer (CRC), in a subject. The method is based on the determination of the level of a panel of least one, preferably 3, 4 and most preferably at least 5, protein biomarker selected from the group consisting of the protein biomarkers Amphiregulin (AREG), Carcinoembryonic antigen (CEA), Insulin like growth factor bindng protein 2 (IGFBP2), Keratin, type I cytoskeletal 19 (KRT19), Mannan binding lectin serine protease 1 (MASP1), Osteopontin (OPN), Serum paraoxonase lactonase 3 (PON3) and Transferrin receptor protein 1 (TR), in the biological sample obtained from the subject. The new biomarker panel of the invention allows diagnosing and even stratifying various cancer diseases. Furthermore, provided are diagnostic kits for performing the non-invasive methods of the invention. Since the biomarker panel of the invention provides a statistically robust method independent of the protein detection technology used, and considering that the biomarker panel of the invention is detected in plasma samples of the subjects, the invention provides an early detection screening examination that may be applied to a larger population.

### DESCRIPTION

A major step in many aspects of research related to diseases such as cancer is the identification of specific and sensitive biomarkers suitable for the development of effective and improved diagnostic, prognostic and therapeutic modalities. An aim of the present invention is to provide novel biomarkers and biomarker panels for use as novel diagnostic and/or prognostic markers and/or for use in the development of novel therapeutics. Whilst mass spectrometry, shot gun proteomics and DNA/RNA microarray analyses, and deep sequencing have resulted in an increasing list of reported potential tumor biomarkers, very few have found their way into the clinical validation phase and even fewer are used as reliable therapeutic targets or diagnostic markers.

Colorectal Cancer (CRC) contributes immensely to the global burden of cancers with 1.85 million incident cases and approximately 880,000 deaths per year. CRC is the third most common cancer and second leading cause of cancer mortality globally [1]. There is increasing evidence that CRC incidence and mortality could be effectively reduced by screening [2-5]. Nevertheless, the participation rates in screening programs are often low due to inconvenience and invasiveness in endoscopy based programs [6, 7] and by reservation against collection, handling and storage of stool in stool test based screening programs [8]. The participation in population based screening programs could potentially improve with minimally invasive blood based tests [9].

The human proteome is estimated to consist of more than 20,000 proteins [10] and is being intensively explored for blood based biomarker research. Multiplex platforms like liquid chromatography-multiple reaction monitoring/mass spectrometry (LC-MRM/MS) [11, 12] and proximity extension assay (PEA) [13, 14] can facilitate accurate simultaneous detection of numerous proteins in one go and have been used for blood based biomarker research. Both LC-MRM/MS and PEA possess the ability of detecting even low abundant markers with analytical sensitivity in the nanogram/ml to picogram/ml range.

Several blood-based protein marker signatures have been identified in previous years but only very few studies validated the signatures in an independent validation set and so far no study has repeated protein measurements in the same set of samples with different protein detection methods [15,16].

Hence the current invention was conceived based on an objective to identify, evaluate and validate a protein biomarker signature for CRC early detection in a three stage design. The proteins were firstly assayed in blood samples of CRC cases and controls using LC-MRM/MS in order to identify a promising multi-marker algorithm. The identified algorithm was then evaluated using PEA, another highly sensitive method in samples from same population. Finally the estimates were independently validated in prospectively collected samples of CRC and advanced adenoma (AA) cases and controls free of colorectal neoplasms that were exclusively recruited in a true screening setting.

Due to the continuing need for quick, but sensitive and specific cancer diagnostics the present invention seeks to provide a novel approach for a simple and minimal invasive but specific and sensitive test system for the diagnosis or monitoring various cancer diseases. In particular the screening of colorectal cancer, which is currently done with procedures highly unpleasant for the patients and associated with discomfort needs new methods in order to convince subjects to undergo CRC screening.

### BRIEF DESCRIPTION OF THE INVENTION

Generally, and by way of brief description, the main aspects of the present invention can be described as follows:

In a first aspect, the invention pertains to a method for the diagnosis, prognosis, stratification and/or monitoring of a therapy, of a cancer disease in a subject, comprising the steps of:
(a) Providing a biological sample from the subject,
(b) Determining the level (concentration) of at least one biomarker selected from the group consisting of the biomarkers Amphiregulin (AREG), Carcinoembryonic antigen (CEA), Insulin like growth factor binding protein 2 (IGFBP2), Keratin, type I cytoskeletal 19 (KRT19), Mannan binding lectin serine protease 1 (MASP1), Osteopontin (OPN), Serum paraoxonase lactonase 3 (PON3) and Transferrin receptor protein 1 (TR), in the biological sample, wherein a differential level of the at least one, preferably, two, three, four and most preferable 5, biomarkers in the biological sample from the subject as determined in step (b) compared to a healthy control or reference value is indicative for the presence of a cancer disease in the subject.

In a second aspect, the invention pertains to a diagnostic kit for performing a method according to the first aspect of the invention.

In a third aspect, the invention pertains to a use of an antibody, or antigen binding fragment thereof, directed to any one of the protein biomarkers selected from TR, OPN, IGFBP2, MASP1, PON3, AREG, CEA and/or KRT19, in the performance of a method according to the first aspect.

In a fourth aspect, the invention pertains to a screening examination method for the early detection of a cancer disease, preferably CRC, in a subject not being diagnosed to have the cancer disease before, the method comprising
(a) Providing a biological sample of the subject to be screened,
(b) Performing a method according to the first aspect with the so provided biological sample of the subject.

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the invention pertains to a method for the diagnosis, prognosis, stratification and/or monitoring of a therapy, of a cancer disease in a subject, comprising the steps of:
(a) Providing a biological sample from the subject,
(b) Determining the level (concentration) of at least one biomarker selected from the group consisting of the biomarkers Amphiregulin (AREG), Carcinoembryonic antigen (CEA), Insulin like growth factor binding protein 2 (IGFBP2), Keratin, type I cytoskeletal 19 (KRT19), Mannan binding lectin serine protease 1 (MASP1), Osteopontin (OPN), Serum paraoxonase lactonase 3 (PON3) and Transferrin receptor protein 1 (TR), in the biological sample,
   wherein a differential level of the at least one, preferably, two, three, four and most preferable 5, biomarkers in the biological sample from the subject as determined in step (b) compared to a healthy control or reference value is indicative for the presence of a cancer disease in the subject.

The term protein biomarker shall refer in context of the invention to a marker which is a protein molecule, preferably a protein which is not an antibody. The biomarkers of the invention are preferably selected from TR, OPN, IGFBP2, MASP1, PON3, AREG, CEA and KRT19. As a reference, the designations of the protein biomarkers are referring to their respective entries in the UniProt database (UniProt: a worldwide hub of protein knowledge Nucleic Acids Research, Volume 47, Issue D1, 8 Jan. 2019, Pages D506-D515; “www.uniprot.org/”) in its version of May 7, 2019. The UniProt identification numbers of the disclosed protein biomarkers are provided herein in table 4 in FIG. 6. By reference, the amino acid sequences of such protein entries of the 8 biomarkers of the invention are incorporated herein by reference.

A “diagnosis” or the term “diagnostic” in context of the present invention means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The term “prognosis” refers to a forecast as to the probable outcome of the disease as well as the prospect of recovery from the disease as indicated by the nature and symptoms of the case. Accordingly, a negative or poor prognosis is defined by a lower post-treatment survival term or survival rate. Conversely, a positive or good prognosis is defined by an elevated post-treatment survival term or survival rate. Usually prognosis is provided as the time of progression free survival or overall survival.

The term “stratification” for the purposes of this invention refers to the advantage that the method according to the invention renders possible decisions for the treatment and therapy of the patient, whether it is the hospitalization of the patient, the use, effect and/or dosage of one or more drugs, a therapeutic measure or the monitoring of a course of the disease and the course of therapy or etiology or classification of a disease, e.g., into a new or existing subtype or the differentiation of diseases and the patients thereof. Particularly with regard to colorectal cancer, “stratification” means in this context a classification of a colorectal cancer as early or late stage colorectal cancer.

The term “monitoring a therapy” means for the purpose of the present invention to observe disease progression in a subject who receives a cancer therapy. In other words, the subject during the therapy is regularly monitored for the effect of the applied therapy, which allows the medical practitioner to estimate at an early stage during the therapy whether the prescribed treatment is effective or not, and therefore to adjust the treatment regime accordingly.

As used herein, the term “subject” or “patient” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject. As used herein, the term “subject suspected of having cancer” refers to a subject that presents one or more symptoms indicative of a cancer (e.g., a noticeable lump or mass). A subject suspected of having cancer may also have one or more risk factors. A subject suspected of having cancer has generally not been tested for cancer. However, a “subject suspected of having cancer” encompasses an individual who has received an initial diagnosis (e.g., a CT scan showing a mass) but for whom the sub-type or stage of cancer is not known. The term further includes people who once had cancer (e.g., an individual in remission), and people who have cancer and are suspected to have a metastatic spread of the primary tumor. In this regard the present invention is also applicable as follow-up care for monitoring a subject for a reoccurrence of the cancer.

The term “cancer” and “cancer cells” refers to any cells that exhibit uncontrolled growth in a tissue or organ of a multicellular organism. Particular preferred cancers in context of the present invention are selected from colorectal cancer, pancreatic cancer, gastric cancer, breast cancer, lung cancer, prostate cancer, hepatocellular cancer, cervical cancer, ovarian cancer, liver cancer, bladder cancer, cancer of the urinary tract, thyroid cancer, renal cancer, carcinoma, melanoma, leukemia or brain cancer.

As used herein, the term “colorectal cancer” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions, which are characterized by cancer of cells of the duodenum and small intestine (jejunum and ileum).

As used herein, the terms “gastric cancer” or “stomach cancer” refer to cancers of the stomach. The most common types of gastric cancer are carcinomas, such as but not limited to, adenocarcinomas, affecting the epithelial cells of the stomach. Stomach cancers may additionally include, for example, sarcomas affecting the connective tissue of the stomach and blastomas affecting the blast tissue of the stomach.

The term “pancreatic cancer” encompasses benign or malignant forms of pancreatic cancer, as well as any particular type of cancer arising from cells of the pancreas (e.g., duct cell carcinoma, acinar cell carcinoma, papillary carcinoma, adenosquamous carcinoma, undifferentiated carcinoma, mucinous carcinoma, giant cell carcinoma, mixed type pancreatic cancer, small cell carcinoma, cystadenocarcinoma, unclassified pancreatic cancers, pancreatoblastoma, and papillary-cystic neoplasm, and the like.).

The term “biological sample” as used herein refers to a sample that was obtained and may be assayed for any one of the biomarkers as disclosed with the present invention, or their gene expression. The biological sample can include a biological fluid (e.g., blood, cerebrospinal fluid, urine, plasma, serum), tissue biopsy, and the like. In some embodiments, the sample is a tissue sample, for example, tumour tissue, and may be fresh, frozen, or archival paraffin embedded tissue. Preferred samples for the purposes of the present invention are bodily fluids, in particular blood or plasma samples.

A “biomarker” or “marker” in the context of the present invention refers to an organic biomolecule, particularly a polypeptide, which is differentially present in a sample taken from subjects having a certain condition as compared to a comparable sample taken from subjects who do not have said condition (e.g., negative diagnosis, normal or healthy subject, or non-cancer patients, depending on whether the patient is tested for cancer, or metastatic cancer). For examples, a marker can be a polypeptide or protein (having a particular apparent molecular weight) which is present at an elevated or decreased level in samples of cancer patients compared to samples of patients with a negative diagnosis. Insofar the invention refers to the determination of the level of protein biomarker, the presence of the respective full length protein, or any fragment thereof, is comprised by the present invention. Fragments preferably have a length sufficient to specifically determine that they are derived from the parent full length protein, such fragments are preferably at least 8, 9, 10, 15, 20, 30, 40, 50 or more amino acids long.

As an alternative the protein biomarkers of the invention may also be detected indirectly by detecting autoantibodies directed to the protein biomarkers.

The term “determining the level of” a biomarker in a sample, control or reference, as described herein shall refer to the quantification of the presence of said biomarkers in the testes sample. For example the concentration of the biomarkers in said samples may be directly quantified via measuring the amount of protein/polypeptide/polysaccharide as present in the tested sample. However, also possible is to quantify the amount of biomarker indirectly via assessing the gene expression of the encoding gene of the biomarker, for example by quantification of the expressed mRNA encoding for the respective biomarker. The present invention shall not be restricted to any particular method for determining the level of a given biomarker, but shall encompass all means that allow for a quantification, or estimation, of the level of said biomarker, either directly or indirectly. “Level” in the context of the present invention is therefore a parameter describing the absolute amount of a biomarker in a given sample, for example as absolute weight, volume, or molar amounts; or alternatively “level” pertains to the relative amounts, for example and preferably the concentration of said biomarker in the tested sample, for example mol/l, g/l, g/mol etc. In preferred embodiments the “level” refers to the concentration of the tested biomarkers in g/l.

“Increase” of the level of a biomarker in a sample compared to a control shall in preferred embodiments refer to statistically significant increase in preferred aspects of the invention.

In alternative embodiments of the invention, certain biomarkers as disclosed herein may also be significantly decreased in the event of a cancer disease in a subject.

In a preferred embodiment the method of the herein disclosed invention is performed non-invasive, such as an in vitro or ex vivo method. So far the herein described diagnostic methods are non-invasive the term “providing a biological” sample shall preferably not be interpreted to include a surgical procedure conducted at the subject.

Preferred embodiments of the present invention pertain to panels of a plurality of biomarkers as identified herein for the diagnostic purposes as described. The advantage of combing the biomarkers, in particular the combination of protein biomarkers and autoantibody biomarkers, as disclosed herein, is an increased sensitivity and/or specificity of the diagnostic assays. Hence a preferred embodiment of the invention pertains to the herein disclosed method wherein step (b) comprises determining the level of at least two, three, four or preferably five, most preferably all eighth biomarkers in the biological sample.

In context of the invention any combination of 4 protein biomarkers of the disclosed list of 8 biomarkers is advantageous and forms part of the invention.

In some particular embodiments, the invention pertains to the method of the first aspect wherein step (b) comprises determining a combination of at least 4 of said biomarkers, preferably (i) MASP1, OPN, PON3 and TR, or (ii) AREG, MASP1, OPN, PON3, and TR, or (iii) AREG, MASP1, OPN, PON3, TR, CEA and KRT19.

In this regard it is preferred that the analysis of a four biomarker panel in step (b) of the diagnostic method of the invention is characterized in that the tested biomarker panel has an apparent area under the curve (AUC) according to FIGS. 7 and 8.

In some preferred embodiments of the invention, step (b) of the method of the first aspect comprises determining the level of at least the protein biomarker TR, OPN, IGFBP2, MASP1, and PON3, in the biological sample (five biomarker panel).

Regarding the five biomarker panel comprising the markers TR, OPN, IGFBP2, MASP1, and PON3, it is preferred that the analysis of the biomarker panel in step (b) of the diagnostic method of the invention is characterized in that the tested marker panel has an apparent area under the curve (AUC) according to FIGS. 4, 5, and 8

In some preferred embodiments of the invention, step (b) of the method of the first aspect comprises determining the level of TR, OPN, IGFBP2, MASP1, and PON3 and further of one or more additional biomarkers selected from the group consisting of AREG, CEA and/or KRT19, in the biological sample.

In some preferred embodiments of the invention, step (b) of the method of the first aspect comprises determining the level of at least the protein biomarker TR, OPN, IGFBP2, MASP1, PON3, AREG, CEA and KRT19, in the biological sample (eight biomarker panel).

In this regard it is preferred that the analysis of the eight biomarker panel in step (b) of the diagnostic method of the invention is characterized in that the tested biomarker panel has an apparent area under the curve (AUC) according to FIG. 5 .

To date, no single blood biomarker qualifying for mass screening has been identified. The combination of multiple markers might be a more promising approach to achieve the necessary sensitivity and specificity for application in mass screening. Although other marker panels were tested in the prior art, the apparent differences to the panel as provided herein can be explained by the fact that those prior art studies were done in a clinical setting and did not apply any adjustment for over-optimism. The above mentioned limitations were also shared by many other studies regarding blood biomarkers for CRC detection. For reasons outlined in detail in the introduction, it is a critical issue to identify and evaluate biomarkers in samples from screening settings in order to obtain valid performance characteristics under screening conditions. Furthermore, as demonstrated herein, correction for overfitting (cross-validation, bootstrap techniques) and/or external validation are also indispensable to adjust for potential overestimation of diagnostic performance. Hence, the marker panel of the present invention is advantageous over previous prior art panels. The panels of biomarkers according to the invention have the surprising technical advantage that they provide a robust diagnostic result independent on the technology of protein detection used. The invention therefore also lies in the fact that similar results are achieved with the biomarker panels of the invention with both PEA and LC-MRM/MS.

The biomarkers of the invention are preferably protein biomarkers.

The biomarker panel as disclosed herein is particular useful in a cancer screening setting. Cancer screening in the herein disclosed invention shall refer to a procedure where a subject is for which not diagnosis was established is tested for the presence of the cancer disease. This shall not be interpreted to exclude the use of the biomarker of the invention for a diagnostic of a subject that was already diagnosed to suffer from a cancer disease. Non limiting examples for such an application are confirmation of a diagnosis, monitoring or treatment success or monitoring reoccurrence of a cancer in a subject that already received a treatment and wherein cancer is in remission or was cured.

The skilled artisan will understand that numerous methods may be used to select a threshold or reference value for a particular marker or a plurality of markers. In diagnostic aspects, a threshold value may be obtained by performing the assay method on samples obtained from a population of patients having a certain type of cancer, and from a second population of subjects that do not have cancer. For prognostic or treatment monitoring applications, a population of patients, all of which have, for example, ovarian cancer, may be followed for the time period of interest (e.g., six months following diagnosis or treatment, respectively), and then dividing the population into two groups: a first group of subjects that progress to an endpoint (e.g., recurrence of disease, death); and a second group of subjects that did not progress to the end point. These are used to establish “low risk” and “high risk” population values for the marker(s) measured, respectively. Other suitable endpoints include, but are not limited to, 5-year mortality rates or progression to metastatic disease.

Once these groups are established, one or more thresholds may be selected that provide an acceptable ability to predict diagnosis, prognostic risk, treatment success, etc. In practice, Receiver Operating Characteristic curves, or “ROC” curves, are typically calculated by plotting the value of a variable versus its relative frequency in two populations (called arbitrarily “disease” and “normal” or “low risk” and “high risk” for example). For any particular marker, a distribution of marker levels for subjects with and without a disease may overlap. Under such conditions, a test does not absolutely distinguish “disease” and “normal” with 100% accuracy, and the area of overlap indicates where the test cannot distinguish “disease” and “normal.” A threshold is selected, above which (or below which, depending on how a marker changes with the disease) the test is considered to be “positive” and below which the test is considered to be “negative.” The area under the ROC curve (AUC) is a measure of the probability that the perceived measurement may allow correct identification of a condition.

Additionally, thresholds may be established by obtaining an earlier marker result from the same patient, to which later results may be compared. In some aspects, the individuals act as their own “control group.” In markers that increase with disease severity or prognostic risk, an increase over time in the same patient can indicate a worsening of disease or a failure of a treatment regimen, while a decrease over time can indicate remission of disease or success of a treatment regimen.

In some embodiments, multiple thresholds or reference values may be determined. This can be the case in so-called “tertile,” “quartile,” or “quintile” analyses. In these methods, the “disease” and “normal” groups (or “low risk” and “high risk”) groups can be considered together as a single population, and are divided into 3, 4, or 5 (or more) “bins” having equal numbers of individuals. The boundary between two of these “bins” may be considered “thresholds.” A risk (of a particular diagnosis or prognosis for example) can be assigned based on which “bin” a test subject falls into.

In a preferred embodiment said sample is selected from the group consisting of body fluids or tissue, preferably wherein said body fluid sample is a blood sample, more preferably a plasma or serum sample.

In all aspects and embodiments of the present invention in may be preferred that the level of said at least one biomarker in said sample is determined by means of a nucleic acid detection method or a protein detection method. However, nucleic acid detection methods are only applicable where an expressed protein is the biomarker. Generally, all means shall be comprised by the present invention which allow for a quantification of the expression of any one of the herein disclosed biomarker. Therefore, also promoter analysis and procedures assessing the epigenetic status of a gene locus encoding a protein biomarker of the invention are comprised by the herein described invention.

Detection methods that are preferred in context of the herein described invention the level of said at least one biomarker in said sample is determined by means of a detection method selected from the group consisting of mass spectrometry, mass spectrometry immunoassay (MSIA), antibody-based protein chips, 2-dimensional gel electrophoresis, stable isotope standard capture with anti-peptide antibodies (SISCAPA), high-performance liquid chromatography (HPLC), western blot, cytometry bead array (CBA), protein immuno-precipitation, radio immunoassay, ligand binding assay, and enzyme-linked immunosorbent assay (ELISA), preferably wherein said protein detection method is ELISA. Suitable alternative detection methods for quantification of a biomarker of the invention are known to the skilled artisan. However, specifically preferred are liquid chromatography-multiple reaction monitoring/mass spectrometry (LC-MRM/MS) and/or Proximity Extension Assay (PEA), for example as referenced herein above.

With regard to autoantibody biomarkers, an immunological capture assay using a protein or protein fragment is preferred. In these assay the autoantibody is detected by detecting the binding of the autoantibody to its respective antigen, or to a fragment of the antigen which contains the binding epitope. Such methods for autoantibody detection are well known to the skilled artisan.

In yet another aspect, the invention provides kits for aiding a diagnosis of cancer, wherein the kits can be used to detect the biomarkers of the present invention. For example, the kits can be used to detect any one or combination of biomarkers described above, which biomarkers are differentially present in samples of a patient having the cancer and healthy patients. The kits of the invention have many applications. For example, the kits can be used to differentiate if a subject has the cancer, or has a negative diagnosis, thus aiding a cancer diagnosis. In another example, the kits can be used to identify compounds that modulate expression of the biomarkers in in vitro cancer cells or in vivo animal models for cancer.

Optionally, the kit can further comprise instructions for suitable operational parameters in the form of a label or a separate insert. For example, the kit may have standard instructions informing a consumer how to wash the probe after a sample of plasma is contacted on the probe.

In another embodiment, a kit comprises (a) an antibody that specifically binds to a marker; and (b) a detection reagent. Such kits can be prepared from the materials, and the previous discussion regarding the materials (e.g., antibodies, detection reagents, immobilized supports, etc.) is fully applicable to this section and need not be repeated.

In either embodiment, the kit may optionally further comprise a standard or control information so that the test sample can be compared with the control information standard to determine if the test amount of a marker detected in a sample is a diagnostic amount consistent with a diagnosis of cancer.

Preferably the kit of the invention is a diagnostic kit for performing a method in accordance with the present invention comprising means for quantifying the level of said at least one biomarker. Preferably the kit of the invention comprises means for quantifying a protein biomarker selected from TR, OPN, IGFBP2, MASP1, PON3, AREG, CEA and KRT19. Such means for quantifying is for example at least one antibody, or antigen binding fragments thereof, preferably wherein the antibody is a monoclonal antibody, such as a monoclonal antibody that specifically binds to any of the aforementioned biomarkers. Such antibodies are known in the art and commercially available. Alternatively, or additionally the diagnostic kit of the invention may contain means for detection of the presence or absence, and quantification thereof, of an autoantibody against any of the herein disclosed protein biomarkers.

Another aspect of the invention then pertains to the use of an antibody, or antigen binding fragment thereof, directed to any one of the protein biomarkers selected from TR, OPN, IGFBP2, MASP1, PON3, AREG, CEA and/or KRT19, in the performance of a method according to the invention.

One additional aspect of the invention pertains to a screening examination method for the early detection of a cancer disease, preferably CRC, in a subject not being diagnosed to have the cancer disease before, the method comprising
(a) Providing a biological sample of the subject to be screened,
(b) Performing a method according to any one of aspects of the invention with the so provided biological sample of the subject.

In some embodiments of the invention the method screening examination is preferred, wherein if the level of the determined biomarkers indicate the presence of the cancer disease, (i) the method is repeated with an independent biological sample provided of the subject, and/or (ii) the subject is scheduled for a secondary diagnosis of the cancer disease. The secondary diagnosis in the event the cancer disease is CRC is for example a colonoscopy.

Also provided are methods of treatment of a patient suspected of suffering from a cancer disease, the method comprising the steps of performing a diagnostic method according to the invention, optionally subsequently performing a secondary validation of the diagnosis, and then treating the subject with a therapy sufficient to alleviate the diagnosed cancer disease.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

The figures show:

FIG. 1 shows a STARD (Standards for Reporting of Diagnostic Accuracy) flow diagram BLITZ-Study; Abbreviations: AA-Advanced Adenoma; CRC-Colorectal Cancer; HPP-Hyperplastic Polyps; NAA-Non advanced Adenoma; NCP-Non classified polyp; SP-Serrated poly.

FIG. 2 shows the comparison of the ROC curves for detecting (all/early/late) stage CRC & AA vs free of neoplasm controls with five and eight marker signatures. Abbreviations: AA-Advanced Adenomas; AUC-Area under the curve; CRC-Colorectal Cancer; ROC-Receiver operating characteristics.

FIG. 3 shows table 1: characteristics of the study population. Abbreviations: AA-Advanced Adenoma; CRC-Colorectal Cancer; N-number; SD-Standard deviation.

FIG. 4 shows table 2: Diagnostic performance of individual protein biomarkers for detecting CRC. Abbreviations: AUC-Area under the Receiver Operating Curve; AUCBS-0.632+ bootstrap estimates of AUC; AUC*-apparent AUC; CRC-Colorectal Cancer; 95% CI-95% Confidence Interval; Se-Sensitivity; Sp-Specificity. All proteins abbreviations: AREG-Amphiregulin; CDH5-Cadherin 5; CEA-Carcinoembryonic antigen; Gal 3-Galectin 3; IGFBP2-Insulin like growth factor binding protein 2; KRT19-Keratin, type I cytoskeletal 19; MASP1-Mannan binding lectin serine protease 1; MMP9-Matrix metalloproteinase 9; MPO-Myeloperoxidase; OPN-Osteopontin; PON3-Serum paraoxonase lactonase 3; PRTN3-Myeloblastin; SPARC-SPARC protein; TR-Transferrin receptor protein 1.

FIG. 5 shows table 3: Diagnostic performance of multi-marker signatures for detecting CRC (all stages/early/late) and AA. Abbreviations: AA-Advanced Adenomas; AUC-Area under the Receiver Operating Curve; AUCBS-0.632+ bootstrap estimates of AUC; AUC*-apparent AUC; CRC-Colorectal Cancer; 95% CI-95% Confidence Interval; Se-Sensitivity; Sp-Specificity. All proteins abbreviations: AREG-Amphiregulin; CEA-Carcinoembryonic antigen; IGFBP2-Insulin like growth factor binding protein 2; KRT19-Keratin, type I cytoskeletal 19; MASP1-Mannan binding lectin serine protease 1; OPN-Osteopontin; PON3-Serum paraoxonase lactonase 3; TR-Transferrin receptor protein 1.

FIG. 6 shows table 4: Functions of proteins from multi-marker signatures. All proteins abbreviations: AREG-Amphiregulin; CEA-Carcinoembryonic antigen; IGFBP2-Insulin like growth factor binding protein 2; KRT19-Keratin, type I cytoskeletal 19; MASP1-Mannan-binding lectin serine protease 1; OPN-Osteopontin; PON3-Paraoxonase 3; TR-Transferrin receptor protein 1.

FIG. 7 shows table 5: Diagnostic performance of multi-marker signatures for detecting CRC (all stages/early/late) and AA. Abbreviations: AA-Advanced Adenomas; AUC-Area under the Receiver Operating Curve; AUCBS-0.632+ bootstrap estimates of AUC; AUC*-apparent AUC; CRC-Colorectal Cancer; 95% CI-95% Confidence Interval; Se-Sensitivity; Sp-Specificity. All proteins abbreviations: AREG-Amphiregulin; CEA-Carcinoembryonic antigen; IGFBP2-Insulin like growth factor binding protein 2; KRT19-Keratin, type I cytoskeletal 19; MASP1-Mannan binding lectin serine protease 1; OPN-Osteopontin; PON3-Serum paraoxonase lactonase 3; TR-Transferrin receptor protein 1.

FIG. 8 shows table 6: Diagnostic performance of multi-marker signatures for detecting CRC (all stages/early/late) and AA. Abbreviations: AA-Advanced Adenomas; AUC-Area under the Receiver Operating Curve; AUCBS-0.632+ bootstrap estimates of AUC; AUC*-apparent AUC; CRC-Colorectal Cancer; 95% CI-95% Confidence Interval; Se-Sensitivity; Sp-Specificity. All proteins abbreviations: AREG-Amphiregulin; CEA-Carcinoembryonic antigen; IGFBP2-Insulin like growth factor binding protein 2; KRT19-Keratin, type I cytoskeletal 19; MASP1-Mannan binding lectin serine protease 1; OPN-Osteopontin; PON3-Serum paraoxonase lactonase 3; TR-Transferrin receptor protein 1.

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

### Example 1: Characteristics of Study Population

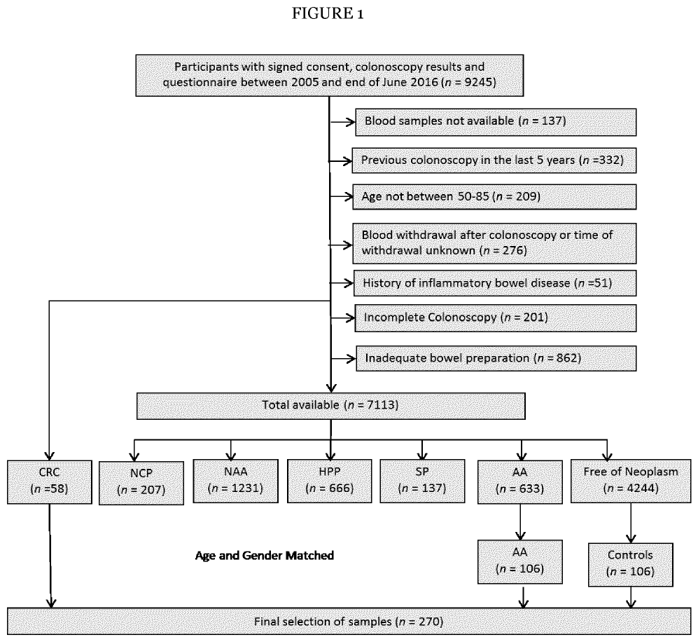

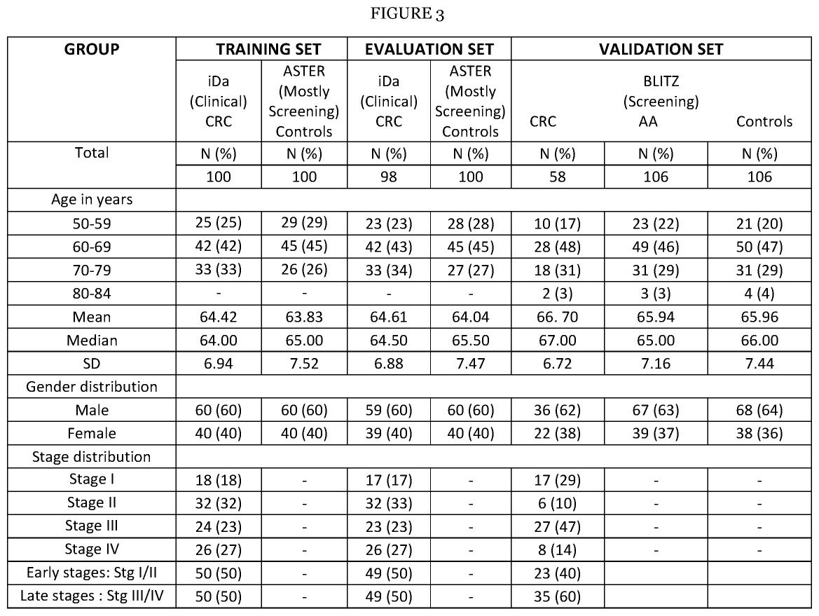

The STARD diagrams displaying selection of study participants enrolled in iDa, ASTER and BLITZ are provided in FIG. 1, respectively. The discovery set A used for LC-MRM/MS consisted of 100 clinically recruited CRC cases and 100 controls free of neoplasms and for discovery set B using PEA of 98 CRC cases and wo controls free of neoplasms, from iDa and ASTER studies, respectively. The three stage specific prediction algorithms were then externally evaluated and validated in a validation set comprising CRC cases and sex and age matched controls without colorectal neoplasms selected from participants of screening colonoscopy. Because the sex and age distribution of participants with AA or those of controls free of neoplasm actually differs from the corresponding distributions among CRC cases in true screening practice, observations from participants with AA and controls without neoplasms were weighted in the analysis in such a way that their sex and age distribution reflects the sex and age distribution of all participants with AA and controls without neoplasms among the participants of screening colonoscopy, respectively, in order to provide valid estimates of the algorithms' performance in a true screening setting. The validation set that consisted of participants of screening colonoscopy consisted of 58 CRC, 106 AA and 106 controls free of neoplasms from the BLITZ study. The characteristics of populations from all three sets are shown in Table 1 in FIG. 3. The distribution of characteristics was largely similar across all three sets with males representing ≥60% of population and median age being around 65 years in all sets. The validation set included a higher proportion of stage I CRCs and a lower proportion of stage IV CRCs than the discovery sets A and B.

### Example 2: Individual Markers

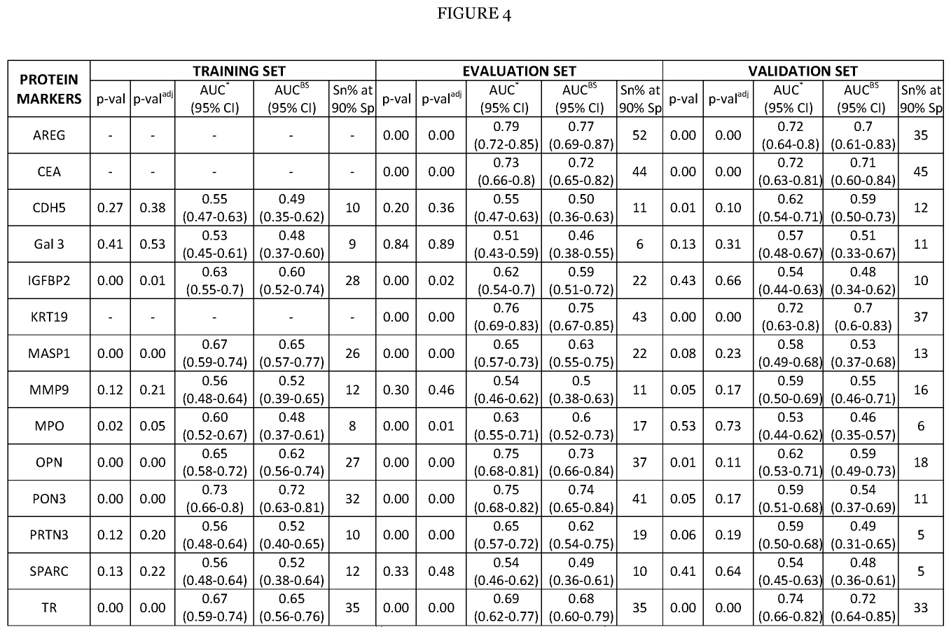

The diagnostic performances of all the above mentioned protein markers across the three different sets are listed in Table 2 in FIG. 4. After adjustment for multiple corrections, the number of proteins with significant differences between CRC and controls were (N=6, 7 and 1) in discovery sets A and B and validation set, respectively. The AUCs ≥0.60 were observed for (N=6, 7 and 3) and sensitivities >25% at 90% specificity for (N=6, 7 and 3) protein biomarkers in discovery sets A and B and validation set, respectively. Of the eleven protein biomarkers commonly quantified by both platforms the best individual diagnostic performance was observed for PON3 in discovery set A with an AUCBS of 0.72 (95% CI, 0.63-0.81), in discovery set B with an AUCBS of 0.74 (95% CI, 0.65-0.84) and in validation set for TR with an AUCBS of 0.72 (95% CI, 0.64-0.85). As seen from Table 2 in FIG. 4 the diagnostic performance of nine out of eleven markers was similar in both discovery sets A and B that had an overlap of 96 CRC cases and 94 controls free of colorectal neoplasms.

### Example 3: Correlation Analysis

The results of the Pearson's product-moment correlation analysis for protein biomarkers measured across the same sample from discovery sets A and B consisting of 190 participants (CRC=96 and controls=94) revealed that the correlation coefficient was highest for PON3 (0.79) and was 0.6 for eight out of eleven biomarkers. The good concordance observed for protein biomarkers not only confirms the diagnostic potential of these markers, but also indicates the robustness of the findings.

### Example 4: Multi-Marker Signatures

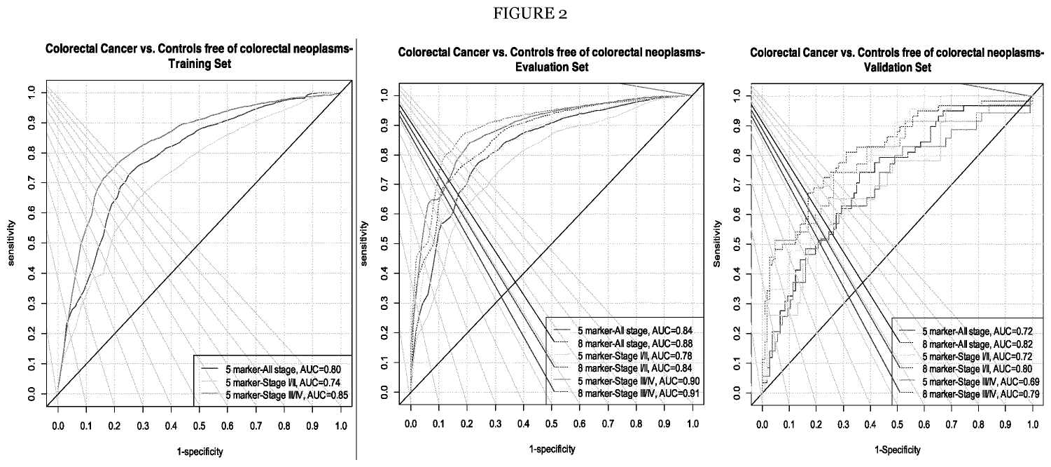

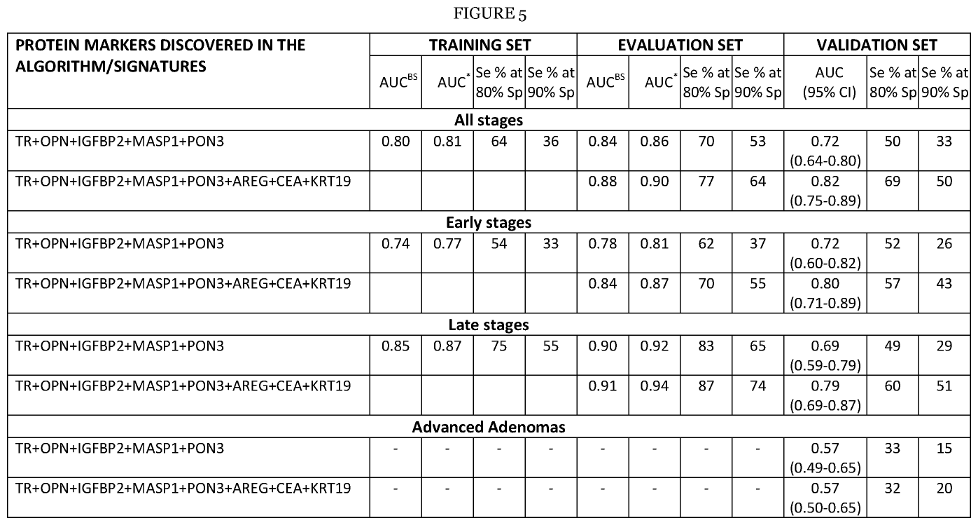

To assess whether the diagnostic performance of individuals protein biomarkers can be improved further, the inventors derived a multi-marker signatures for CRC detection. Table 3 in FIG. 5 and FIG. 2 present an overview upon the diagnostic performance indicators across three sets. For a five marker signature consisting of biomarkers IGFBP2, MASP1, OPN, PON3 and TR, he AUCsBS for all stage CRC detection were 0.80 and 0.84 in the discovery set A and B, respectively, and in the validation set an AUC of 0.72 (95% CI, 0.64-0.80), was observed. For early stage CRC detection, the five marker signature yielded AUCsBS of 0.74, 0.78 and an AUC of 0.72 (95% CI, 0.60-0.82) in discovery sets A and B and validation set, respectively. For late stage CRC detection the AUCsBS were 0.85 and 0.90 in discovery sets A and B, respectively and an AUC of 0.69 (95% CI, 0.59-0.79) was observed in the independent validation set.

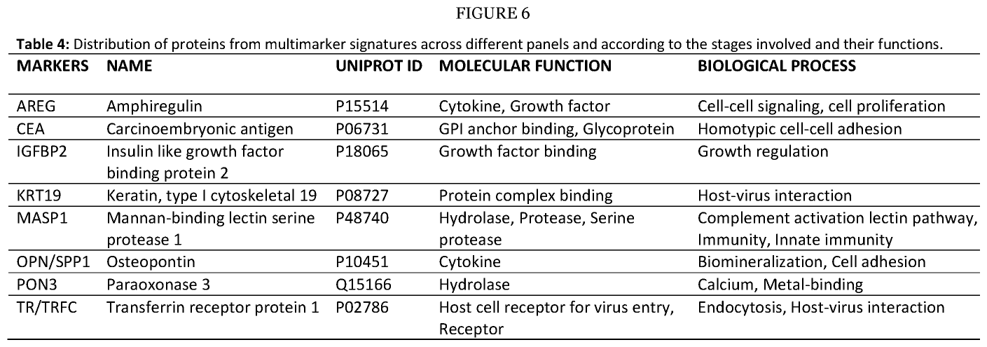

When the diagnostic performance of the proteins from the five marker signature was analyzed in combination with three additional biomarkers AREG, CEA and KRT19 the performance improved (FIG. 5). In the discovery set B for the eight marker signature (AREG, CEA, IGFBP2, KRT19, MASP1, OPN, PON3 and TR), the AUCsBS improved to 0.88, 0.84 and 0.91 for all, early and late CRC detection. In an independent validation set comprising of participants of screening colonoscopy AUCs of 0.82 (95% CI, 0.75-0.89), 0.80 (95% CI, 0.71-0.89) and 0.79 (95% CI, 0.69-0.87) were observed for all, early and late stage CRC detection comparison, respectively. For the eight marker signature in the validation set, the sensitivities at 80% specificity were 69%, 57% and 66% and sensitivities at cutoffs yielding 90% specificity were 50%, 43% and 51% for all, early and late stage CRC detection, respectively. When the five and eight marker signatures were applied for detection of AA cases the same AUC of 0.57 (95% CI, 0.49-0.65) was observed for both signatures. The incorporation of age and gender into signatures did not result in any change in the diagnostic performance. As described in Table 4 in FIG. 6, all the eight biomarkers selected in both signatures had a wide variety of biological and molecular functions. Additionally, when Ingenuity Pathway Analysis (QIAGEN Inc., https://www.qiagenbioinformatics.com/products/ingenuity-pathway-analysis, version 01-10; Ingenuity Systems, Redwood City, USA) was used to understand the interaction between these proteins, it was observed that though functionally different these proteins interact at cellular or extracellular level in development of carcinoma.

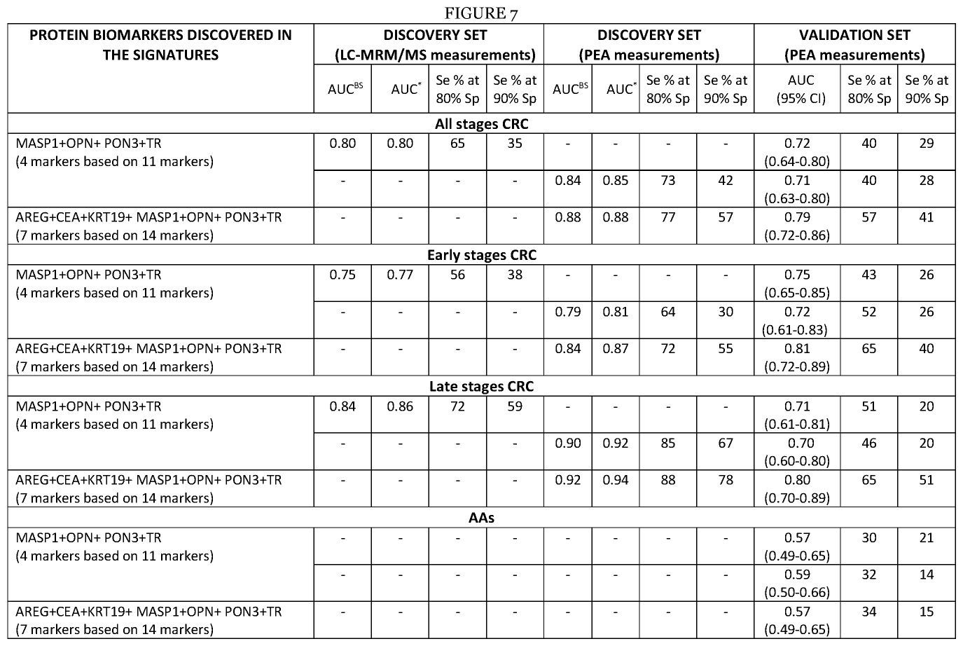

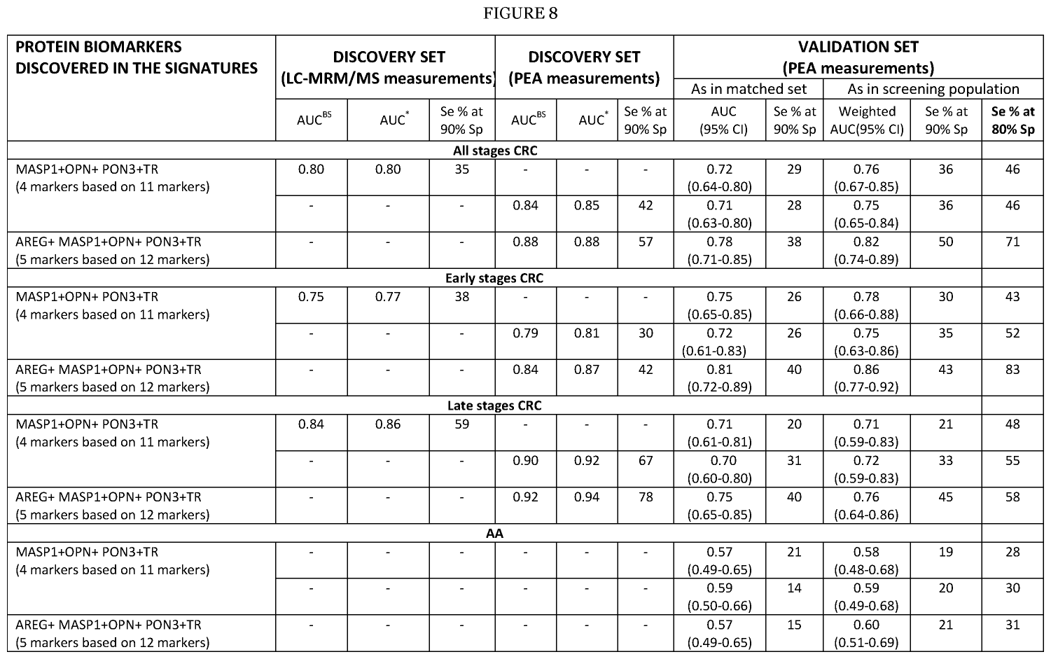

Additional preferably diagnostic algorithms could be calculated with similarly advantageous AUC as shown in FIGS. 7 and 8.

In FIG. 8, the diagnostic performance of a four marker signature comprising the combination of MASP1, OPN, PON3 and TR was analysed and compared to the five marker signature comprising the combination of AREG, MASP1, OPN, PON3 and TR. In the discovery set analysed by LC-MRM/MS measurements, the AUCs^{BS} for the four marker signature (MASP1, OPN, PON3 and TR) measured were 0.80, 0.75, and 0.84 for all, early and late CRC detection. The discovery set was additionally analysed by PEA measurements. When the discovery set was analysed by PEA measurements, the AUCs^{BS} measured for the four marker signature (MASP1, OPN, PON3 and TR), were 0.84, 0.79, and 0.90 for all, early and late CRC detection, compared to AUCs^{BS} for the five marker signature (AREG, MASP1, OPN, PON3 and TR) of 0.88, 0.84 and 0.92 for all, early and late CRC detection.

In an independent validation set comprising participants of screening colonoscopy, AUCs of 0.76 (95% CI, 0.67-0.85), 0.78 (95% CI, 0.66-0.88) and 0.71 (95% CI, 0.59-0.83) were observed for the four marker signature (MASP1, OPN, PON3 and TR) for all, early and late stage CRC detection comparison, when analysed by LC-MRM/MS measurements. When the independent validation set comprising participants of screening colonoscopy was analysed by PEA measurements, AUCs of 0.75 (95% CI, 0.65-0.84), 0.75 (95% CI, 0.63-0.86) and 0.72 (95% CI, 0.59-0.83) were observed for the four marker signature (MASP1, OPN, PON3 and TR) for all, early and late stage CRC detection comparison, compared to AUCs^{BS} for the five marker signature (AREG, MASP1, OPN, PON3 and TR) of 0.82 (95% CI, 0.74-0.89), 0.86 (95% CI, 0.77-0.92) and 0.76 (95% CI, 0.64-0.86) for all, early and late CRC detection.

For the four marker signature (MASP1, OPN, PON3 and TR) in the validation set, the sensitivities at 80% specificity were 46%, 43% and 48% when analysed by LC-MRM/MS measurements. When analysed by PEA measurements, the sensitivities for the four marker signature (MASP1, OPN, PON3 and TR) at 80% specificity were 46%, 52% and 55%, compared to 71%, 83% and 58% as analysed for the five marker signature (AREG, MASP1, OPN, PON3 and TR). Moreover, sensitivities at cutoffs yielding 90% specificity were 36%, 30% and 21% for all, early and late stage CRC detection for the four marker signature (MASP1, OPN, PON3 and TR) when analysed by LC-MRM/MS measurements, and 36%, 35% and 33% for all, early and late stage CRC detection for the four marker signature (MASP1, OPN, PON3 and TR) when analysed by PEA measurements. As a comparison, the sensitivities at cutoffs yielding 90% specificity analysed for the five marker signature (AREG, MASP1, OPN, PON3 and TR) were 50%, 43% and 45% for all, early and late stage CRC detection, when analysed by PEA measurements. The data derived from the independent validation set comprising participants of screening colonoscopy AUCs is a representation of how the markers would perform in the general screening population, where the cases and controls are not matched by age and gender.

Conclusion: In recent years several studies have identified blood based protein marker panels and signatures, which have shown the potential to yield AUCs higher than 0.8 for CRC detection. However, the participants in these studies were not recruited in screening settings [15, 16]. Two previous research studies from the inventors have identified eight and six marker panels with AUCs for CRC of 0.76 [13] and 0.84 [27], respectively. However unlike the current study the CRC cases were validation sets had clinically recruited in those studies. Two other publications where external validation comprised of only preclinical samples yielded AUC for CRC detection of 0.59 [28] and 0.82 [14] for two individual 5-marker signatures. In the current study of the invention it could be included that CRC and AA cases from participants undergoing screening colonoscopy and the number of CRC cases in the validation set is higher than in the previous studies.

Additionally, the proteins in the current study were separately measured with two highly target specific proteomic technologies. Previous research on blood based biomarkers other than proteins like COLOX (gene expression of 29 genes), COLODETECT (4 proteins+3 phages) and CANCERSEEK (16 genes+8 proteins) had CRC cases that were recruited in partial or complete clinical settings and the diagnostic performance of these tests for early detection of CRC in screening setting samples is not known. For COLOSENTRY (7 genes) when the performance was evaluated in screening setting sensitivity of 61% at 77% specificity was observed for all stages. The eight marker algorithm identified according to the invention yielded 57% sensitivity at cutoff yielding 80% specificity. The blood based test Epi proColon 2.0 based on Sept9gene methylation, showed 59% sensitivity at 79% specificity for early stages CRC [29], which is comparable with the diagnostic performance displayed by the eight marker signature from the current study. Therefore, the diagnostic performance of the current signature is in line with results of handful studies validating diagnostic performance of blood-based tests in true screening-setting like the PRESEPT clinical trial on Sept9 gene methylation [30].

The eight proteins identified from both signatures as demonstrated Table 4 in FIG. 6 are involved in different biological processes and also have diverse molecular function. Cytokines AREG and OPN and glycoprotein CEA have been identified previously as biomarkers with diagnostic potential for CRC [13, 14, 27, 29, 31-33]. Protein biomarkers KRT19 and TR which are both involved in biological processes of host-virus interaction and hydrolases PON3 and MASP1 have been recently associated with CRC detection. IGFBP2 a growth factor binding protein is involved in growth regulation. Therefore, the current panel of the invention is nonobvious and surprising and shows the potential of these eight biomarkers as a signature for early detection of CRC with end validation in an independent set consisting of samples from participants of colonoscopy.

The detection and quantitation of low abundant biomarkers with low sample volume has been possible because of advancement in the field of proteomics. The peptides selected for the LC-MRM/MS had good mass spectrometer (MS) responses and uniquely identified the target protein. Moreover, using the triple-quadrupole MS high specificity is achieved firstly by only allowing a selected peptide to pass through the first quadrupole and enter the collision cell where the peptide dissociates into fragments specific to the amino acid sequence of the precursor peptide. Another second stage of technical specificity is added in the second MS, and only a specific fragment is allowed to pass through and strike the detector. Similarly, for PEA the pair of oligonucleotide labeled antibodies or probes have to be in close proximity and only this dual recognition of the target protein leads to initiation of an amplified signal detection. On account of these factors both LC-MRM/MS and PEA are very target specific method. The technical assay sensitivity of the LC-MRM/MS is in mid-high nanogram/ml range and it was obvious practical to use detection method with same ability. Often it is recommended to use ELISA for repeat measurements but individual ELISAs would have required more sample volume than any multiplex platform. Therefore, PEA with analytical sensitivity in picogram/ml was used for repeat measurements. Since both the technologies are highly sensitive and the performance of the biomarkers was almost similar in both discovery sets A and B, it can be certainly determined that the observed diagnostic performances were not simply a matter of chance. Apart from advanced technology used for detection and quantitation, the pre-analytical processing of samples has been shown to influence the measurements in the protein biomarker research [34-36]. In the current study, even though the participants were selected from three different studies the collection, handling, processing and storage of the samples across the three studies were performed with similar standardized operating procedure (SOPs).

This is the first study that identifies, evaluates and validates biomarkers across two different platforms using a three stage design. In the current study the inventors not only performed correction of over-optimism with 0.632+ bootstarp method but also externally validated the findings in an independent validation set that consisted of participants with CRC, AA and no colorectal neoplasms at all at screening colonoscopy. Furthermore, the inventors have re-measured the identified protein biomarkers on two different independent detection methods (LC-MRM/MS and PEA), which are both highly sensitive, target specific technologies and possess the ability of detecting even low abundant markers using very low volumes of plasma. The diagnostic performance of the eight marker signature was fairly good for a blood-based test, with an AUC of 0.82 (95% CI 0.75-0.89) for all stage CRC detection. Therefore, the identified plasma protein biomarkers are potential candidates for further research on blood-based test for CRC screening and early detection.

Utilizing two competitive target specific protein detection methods the invention identified a promising eight marker signature with diagnostic potential for early detection of CRC. The protein biomarkers AREG, CEA, IGFBP2, KRT19, MASP1, OPN, PONS and TR exhibited diagnostic performance competitive with all existing tests comprising of only protein or any other biomarkers validated in true screening setting samples. The biomarkers identified constitute a promising blood-based test for population based screening and early detection of CRC and its premalignant lesions.

### Materials and Methods

### Study Design

The protein biomarkers were assessed in a three-step approach, with first measurement performed in discovery set A using LC-MRM/MS. This was followed by re-evaluating the performance in samples from the same study population in discovery set B using PEA and lastly the algorithm was validated in an independent study population of participants of screening colonoscopy in validation set using PEA again.

### Study Population: Discovery Sets A and B

The discovery set A included 100 CRC cases recruited prior to any therapeutic intervention from the iDa (“Durch innovative Testverfahren Darmkrebs früher erkennen”) study in hospitals in southwestern Germany between 2013 and 2016. As controls the inventors included 100 participants of screening colonoscopy who were recruited in the ASTER (“Mit ASS Darmtumore früher erkennen”) study and were free of colorectal neoplasms. ASTER is a multicenter prospective randomized controlled trial (EudraCT No. 2011-005603-32). Participants of ASTER were recruited and blood samples were taken at recruitment from gastrointestinal practices in Germany from 2013 to 2016 [17]. The discovery set B consisted of 98 CRC cases from the iDa study and 100 controls free of neoplasm from the ASTER study. The study population was nearly the same between both discovery sets (96 CRC cases and 94 controls overlapped between discovery sets A and B) and the difference of ten participants was on account of limited sample volume. The use of samples for early detection of CRC has been approved by the ethics committees of the Medical Faculty Heidelberg and from the responsible state medical boards, for both iDa and ASTER studies.

### Study Population: Validation Set

Blood samples for independent external validation of the algorithm were selected from participants of screening colonoscopy collected in the BLITZ (“Begleitende Evaluierung innovativer Testverfahren zur Darmkrebs-Früherkennung”) study. Details of the BLITZ study design have been reported previously [13, 14, 18-20], briefly, BLITZ is an ongoing prospective screening study of participants of the German screening colonoscopy program that is offered to average risk population. Participants are recruited in 20 gastroenterology practices since end of the year 2005. By the end of June 2016, out of 9425 participants in BLITZ, CRC and AA had been detected in 64 and 633 participants, respectively. In the current study, validation of signatures identified and evaluated in discovery sets A and B, respectively, were carried out in blood samples from 58 participants with CRC and 106 participants that were free of colorectal neoplasm at screening colonoscopy. Additionally, the inventors enriched the study population with 106 participants with AA (defined as adenoma with >1 cm in diameter, tubulovillous or villous components, or highgrade dysplasia [21]. The controls free of neoplasms and also AA participants were frequency matched to the CRC cases by sex and age. The use of samples from the BLITZ study for evaluation of early detection markers for CRC has been approved by the ethics committees of the Medical Faculty Heidelberg (S-178/2005), and of the physicians' boards of Baden-Wuerttemberg (M118-05-f), Rheinland-Palatinate (837.047.06(5145)), and Saarland (217/13). The STARD diagram showing selection of study participants from the BLITZ study is presented in FIG. 1.

### Sample Collection and Storage

Ethylenediaminetetraacetic acid (EDTA) plasma samples were collected before screening colonoscopy in ASTER and BLITZ and at first diagnosis of CRC before any treatment for cancer in iDa. After blood draw, plasma samples were centrifuged between 2000-2500 g for 10 minutes at4° C. Then they were transported to the biobank at German Cancer Research Centre (DKFZ) in a cold chain, centrifuged again, aliquoted, and stored at −80° C. until the protein measurements. All the laboratory analyses were performed blinded with respect to disease status or findings at colonoscopy.

### Laboratory Assays

Plasma samples were analyzed in the discovery set A for the targeted quantitation by peptide based analysis using LC-MRM/MS for eleven proteins that overlapped between both methods, namely, Cadherin 5 (CDH5), Galectin 3 (Gal 3), Insulin like growth factor binding protein 2 (IGFBP2), Mannan binding lectin serine protease 1 (MASP1), Matrix metalloproteinase 9 (MMP9), Myeloperoxidase (MPO), Osteopontin (OPN), Serum paraoxonase lactonase 3 (PON3), Myeloblastin (PRTN3), SPARC protein (SPARC) and Transferrin receptor protein 1 (TR). These peptides had been previously validated for their use in experiments following the Clinical Proteome Tumor Analysis Consortium (CPTAC) guidelines for assay development (“https://assays.cancer.gov”) and the details of the LC-MRM/MS has been published elsewhere [11, 22].

In the discovery set B and validation set protein concentrations in plasma samples were measured utilizing the PEA offered by Olink [23]. Apart from the aforementioned overlapping eleven proteins that were quantified by LC-MRM/MS assay, additionally, three proteins that have been identified promising for early detection of CRC from previous research [13, 14] (also submitted Bhardwaj et al., 2019) namely Amphiregulin, (AREG), Carcinoembryonic antigen (CEA) and Keratin, type I cytoskeletal 19 (KRT19) were analyzed.

### Statistical Analysis

The data from LC-MRM/MS was visualized and examined with Skyline Quantitative Analysis software and the standard curve was used to calculate the peptide concentration in fmol/μl of plasma in the samples. The protein concentrations obtained from PEA were presented in from normalized protein expression (NPX).

The protein values obtained from both LC-MRM/MS and PEA were first compared for each individual biomarker between CRC and controls using Wilcoxon rank-sum test and correction for multiple testing by the Benjamini and Hochberg method [24]. For each individual protein area under the receiver operating characteristic (ROC) curves (AUCs) and their 95% confidence intervals (95% CI) and sensitivity at 8o% and 90% specificities were calculated. Additionally, in order to see concordance between the proteins measured on exactly same samples in discovery sets A and B the Pearson's product-moment correlation was calculated.

In order to measure the diagnostic performance of multi-markers combinations for detection of CRC, LASSO (Least absolute shrinkage and selection operator) regression models with 0.632+ bootstrap [25] to adjust for overfitting, were applied to protein biomarkers in the discovery set A. The biomarkers obtained from the LASSO logistic regression model were further re-evaluated using logistic regression in the discovery set B. Another prediction algorithm was evaluated by combining biomarkers from LASSO regression and AREG, CEA and KRT19. Both the prediction algorithms were externally evaluated in the validation set that exclusively included participants of screening colonoscopy. The diagnostic performance was evaluated by calculating sensitivity at 80% and 90% specificities and apparent AUC i.e. the AUC not adjusted for overfitting (AUC*) with 95% CI, as well as 0.632+ bootstrap adjusted AUC (AUC^{BS}) in discovery sets A and B. All statistical analyses were performed with statistical software R language and environment (version 3.5.0, R core team) [26]. For all tests two-sided p-values of 0.05 or less were considered to be statistically significant.

### REFERENCES

The references are:
1. Bray, F., et al., Global cancer statistics 2018: GLOBOCAN estimates of incidence and mortality worldwide for 36 cancers in 185 countries. C A Cancer J Clin, 2018.
2. Brenner, H., C. Stock, and M. Hoffmeister, Effect of screening sigmoidoscopy and screening colonoscopy on colorectal cancer incidence and mortality: systematic review and meta-analysis of randomised controlled trials and observational studies. BMJ, 2014. 348: p. g2467.
3. Zauber, A. G., The impact of screening on colorectal cancer mortality and incidence: has it really made a difference? Dig Dis Sci, 2015. 60(3): p. 681-91.
4. Atkin, W., et al., Long term effects of once-only flexible sigmoidoscopy screening after 17 years of follow-up: the UK Flexible Sigmoidoscopy Screening randomised controlled trial. The Lancet, 2017. 389(10076): p. 1299-1311.
5. Brenner, H. and C. Chen, The colorectal cancer epidemic: challenges and opportunities for primary, secondary and tertiary prevention. British Journal of Cancer, 2018. 119(7): p. 785-792.
6. Klabunde, C., et al., Participation rates for organized colorectal cancer screening programmes: an international comparison. J Med Screen, 2015. 22(3): p. 119-26.
7. Bretthauer, M., et al., Population-based colonoscopy screening for colorectal cancer: a European randomized trial. JAMA internal medicine, 2016. 176(7): p. 894-902.
8. Robertson, D. J., et al., Recommendations on Fecal Immunochemical Testing to Screen for Colorectal Neoplasia: A Consensus Statement by the US Multi-Society Task Force on Colorectal Cancer. Gastroenterology, 2017. 152(5): p. 1217-1237.e3.
9. Adler, A., et al., Improving compliance to colorectal cancer screening using blood and stool based tests in patients refusing screening colonoscopy in Germany. BMC Gastroenterol, 2014. 14: p. 183.
10. Ponomarenko, E. A., et al., The Size of the Human Proteome: The Width and Depth. International journal of analytical chemistry, 2016. 2016: p. 7436849-7436849.
11. Kuzyk, M. A., et al., Multiple reaction monitoring-based, multiplexed, absolute quantitation of 45 proteins in human plasma. Mol Cell Proteomics, 2009. 8(8): p. 1860-77.
12. Domanski, D., et al., MRM-based multiplexed quantitation of 67 putative cardiovascular disease biomarkers in human plasma. Proteomics, 2012. 12(8): p. 1222-43.
13. Chen, H., et al., Head-to-Head Comparison and Evaluation of 92 Plasma Protein Biomarkers for Early Detection of Colorectal Cancer in a True Screening Setting. Clin Cancer Res, 2015. 21(14): p. 3318-26.
14. Chen, H., et al., Development and validation of a panel of five proteins as blood biomarkers for early detection of colorectal cancer. Clinical Epidemiology, 2017. 9: p. 517-526.
15. Bhardwaj, M., et al., Cell Line Secretome and Tumor Tissue Proteome Markers for Early Detection of Colorectal Cancer: A Systematic Review. Cancers (Basel), 2017. 9(11).
16. Bhardwaj, M., et al., Blood-Based Protein Signatures for Early Detection of Colorectal Cancer: A Systematic Review. Clin Transl Gastroenterol, 2017. 8(11): p. e128.
17. Tikk, K., et al., Clinical trial protocol of the ASTER trial: a double-blind, randomized, placebo-controlled phase III trial evaluating the use of acetylsalicylic acid (ASA) for enhanced early detection of colorectal neoplasms. BMC Cancer, 2018. 18(i): p. 914.
18. Hundt, S., U. Haug, and H. Brenner, Comparative evaluation of immunochemical fecal occult blood tests for colorectal adenoma detection. Ann Intern Med, 2009. 150(3): p. 162-9.
19. Brenner, H., S. Tao, and U. Haug, Low-dose aspirin use and performance of immunochemical fecal occult blood tests. Jama, 2010. 304(22): p. 2513-20.
20. Gies, A., et al., Direct Comparison of Diagnostic Performance of 9 Quantitative Fecal Immunochemical Tests for Colorectal Cancer Screening. Gastroenterology, 2018. 154(1): p. 93-104.
21. Brenner, H., et al., Protection from right- and left-sided colorectal neoplasms after colonoscopy: population-based study. J Natl Cancer Inst, 2010. 102(2): p. 89-95.
22. Percy, A. J., et al., Absolute quantitation of proteins in human blood by multiplexed multiple reaction monitoring mass spectrometry. Methods Mol Biol, 2013. 1000: p. 167-89.
23. Assarsson, E., et al., Homogenous 96-Plex PEA Immunoassay Exhibiting High Sensitivity, Specificity, and Excellent Scalability. PLoS ONE, 2014. 9(4): p. e95192.
24. Benjamini, Y. and Y. Hochberg, CONTROLLING THE FALSE DISCOVERY RATE—A PRACTICAL AND POWERFUL APPROACH TO MULTIPLE TESTING. Journal of the Royal Statistical Society Series B-Methodological, 1995. 57(1): p. 289-300.
25. Efron, B. and R. Tibshirani, Improvements on Cross-Validation: The 632+ Bootstrap Method. Journal of the American Statistical Association, 1997. 92(438): p. 548-56o.
26. Team, R. C., A Language and Environment for Statistical Computing. 2016, R Foundation for Statistical Computing: Vienna, Austria.
27. Surinova, S., et al., Prediction of colorectal cancer diagnosis based oncirculating plasma proteins. Embo Molecular Medicine, 2015. 7(9): p. 1166-1178.
28. Qian, J., et al., Biomarker discovery study of inflammatory proteins for colorectal cancer early detection demonstrated importance of screening setting validation. Journal of Clinical Epidemiology, 2018. 104: p. 24-34.
29. Potter, N. T., et al., Validation of a real-time PCR-based qualitative assay for the detection of methylated SEPT9 DNA in human plasma. Clin Chem, 2014. 60(9): p. 1183-91.
30. Church, T. R., et al., Prospective evaluation of methylated SEPT9 in plasma for detection of asymptomatic colorectal cancer. Gut, 2014. 63(2): p. 317-325.
31. Lumachi, F., et al., Simultaneous multianalyte immunoassay measurement of five serum tumor markers in the detection of colorectal cancer. Anticancer Res, 2012. 32(3): p. 985-8.
32. Werner, S., et al., Evaluation of a 5-Marker Blood Test for Colorectal Cancer Early Detection in a Colorectal Cancer Screening Setting. Clin Cancer Res, 2016. 22(7): p. 1725-33.
33. Wild, N., et al., A combination of serum markers for the early detection of colorectal cancer. Clin Cancer Res, 2010. 16(24): p. 6111-21.
34. Lee, D. H., et al., Proteomic Analysis of the Effect of Storage Temperature on Human Serum. Annals of Clinical & Laboratory Science, 2010. 40(1): p. 61-70.
35. Enroth, S., et al., Effects of Long-Term Storage Time and Original Sampling Month on Biobank Plasma Protein Concentrations. EBioMedicine, 2016. 12: p. 309-314.
36. Shen, Q., et al., Strong impact on plasma protein profiles by precentrifugation delay but not by repeated freeze-thaw cycles, as analyzed using multiplex proximity extension assays. Clin Chem Lab Med, 2018. 56(4): p. 582-594.
37. Gies, A., et al., Quantitative fecal immunochemical tests for colorectal cancer screening. Int J Cancer, 2018. 143(2): p. 234-244.

1. A method for the diagnosis, prognosis, stratification and/or monitoring of a therapy, of a cancer disease in a subject, comprising the steps of:
(a) Providing a biological sample from the subject,
(b) Determining the level (concentration) of at least one (2, 3, 4, 5, 6, or 7 or more) protein biomarker selected from the group consisting of the protein biomarkers Amphiregulin (AREG), Carcinoembryonic antigen (CEA), Insulin like growth factor binding protein 2 (IGFBP2), Keratin, type I cytoskeletal 19 (KRT19), Mannan binding lectin serine protease 1 (MASP1), Osteopontin (OPN), Serum paraoxonase lactonase 3 (PON3) and Transferrin receptor protein 1 (TR), in the biological sample,
wherein a differential level of the at least one, preferably, two, three, four and most preferable 5, biomarkers in the biological sample from the subject as determined in step (b) compared to a healthy control or reference value is indicative for the presence of a cancer disease in the subject.

2. The method according to claim 1, wherein step (b) comprises determining a combination of at least 4 of said biomarkers, preferably (i) MASP1, OPN, PON3 and TR, or (ii) AREG, MASP1, OPN, PON3, and TR, or (iii) AREG, MASP1, OPN, PON3, TR, CEA and KRT19.

3. The method according to claim 1 or 2, wherein step (b) comprises determining the level of at least the protein biomarker TR, OPN, IGFBP2, MASP1, and PON3, in the biological sample.

4. The method according to claim 3, wherein step (b) comprises determining the level of one or more additional biomarkers selected from the group consisting of AREG, CEA and/or KRT19, in the biological sample.

5. The method according to claim 4, wherein step (b) comprises determining the level of at least the protein biomarker TR, OPN, IGFBP2, MASP1, PON3, AREG, CEA and KRT19, in the biological sample.

6. The method according to any of claims 1 to 5, wherein the biological sample is a tissue sample or body liquid sample, preferably a blood sample, most preferably a plasma sample.

7. The method according to any of claims 1 to 6, wherein the method is a non-invasive method, preferably an ex vivo method or in vitro method.

8. The method according to any of claims 1 to 5, wherein the method is a screening method for establishing a first diagnosis of cancer in the subject.

9. The method according to any of the preceding claims wherein the cancer is colorectal cancer, gastric cancer or pancreatic cancer, and preferably is an early stage or late stage CRC.

10. The method according to any one of the preceding claims, wherein the level of said biomarker is determined using one or more antibodies specific for one or more of the respective biomarker proteins, preferably wherein the protein biomarker is detected by western blot, ELISA, Proximity Extension Assay, or mass-spectrometrically, and most preferably is detected by liquid chromatography-multiple reaction monitoring/mass spectrometry (LC-MRM/MS) and/or Proximity Extension Assay (PEA).

11. A diagnostic kit for performing a method according to any of the preceding claims.

12. The diagnostic kit of claim 11, comprising one or more antibodies, or antigen binding fragments thereof, for the detection of the at least one biomarker.

13. Use of an antibody, or antigen binding fragment thereof, directed to any one of the protein biomarkers selected from TR, OPN, IGFBP2, MASP1, PONS, AREG, CEA and/or KRT19, in the performance of a method according to any of claims 1 to 10.

14. A screening examination method for the early detection of a cancer disease, preferably CRC, in a subject not being diagnosed to have the cancer disease before, the method comprising
(a) Providing a biological sample of the subject to be screened,
(b) Performing a method according to any one of claims 1 to 12 with the so provided biological sample of the subject.

15. The method according to claim 14, wherein if the level of the determined biomarkers indicate the presence of the cancer disease, (i) the method is repeated with an independent biological sample provided of the subject, and/or (ii) the subject is scheduled for a secondary diagnosis of the cancer disease.
